(19) United States
(12) Patent Application Publication
(10) Pub. No.: US 2004/0071803 A1
(43) Pub. Date: Apr. 15, 2004

(54) Process for preparation of anti-diabetic compositions from banyan bark

(76) Inventors: Pothapragada Suryanarayana Murthy, Noida (IN); Babu V. Bassa, Santee, CA (US); Krishna Madhava Prabhu, Delhi (IN)

Correspondence Address: KNOBBE MARTENS OLSON & BEAR LLP, 2040 MAIN STREET, FOURTEENTH FLOOR, IRVINE, CA, 92614, US

(21) Appl. No.: 10/350,171

(22) Filed: Jan. 21, 2003

Related U.S. Application Data
(60) Provisional application No. 60/418,390, filed on Oct. 11, 2002.

(51) Int. Cl. A61K 35/78

(52) U.S. Cl. 424/769

(57) ABSTRACT

The present invention is directed in part towards methods of preparing a blood glucose-lowering extract from banyan bark, method of treating a mammal with a high blood glucose level using a banyan bark extract, and pharmaceutical compositions comprising banyan bark extracts.

### BACKGROUND OF THE INVENTION

There are two major types of diabetes mellitus—insulin dependent diabetes mellitus (IDDM) or type I and non-insulin dependent diabetes mellitus (NIDDM) or type II. Of these two, the incidence of NIDDM is much higher.

Present view seems to be that IDDM is due to genetically destructive mechanisms with circulating antibodies to insulin which are aggravated by external factors such as infection (viruses etc.), toxic chemicals etc. It occurs mostly in young patients. For this reason it is also referred to as juvenile diabetes sometimes. As the name suggests insulin is essential for the treatment of IDDM. Oral diabetic drugs are of limited or no use to such patients.

NIDDM occurs mostly in adults, either obese or non-obese. It is also referred to sometimes as maturity onset diabetes. In this condition there is deficiency of insulin secretion or action. This could be due to down regulation of insulin receptors or receptor mediated decreased sensitivity to insulin action or post receptor defect to insulin action. About 70% of the diabetics of the World are estimated to have NIDDM, who need oral antidiabetic drugs either alone or in some cases with insulin.

Drugs Available at Present for the Treatment of Diabetes.

Insulin

It is to be injected and used for type I or non-obese type II diabetics with insulinopenia who do not respond to diet or oral drugs or both. Some of the possible side effects are—insulin antibody formation, insulin resistance or allergic symptoms (less with human insulin).

Oral Drugs

They are also called hypoglycemic drugs because they bring down the higher blood glucose levels seen in diabetic patients. They are broadly of two types: sulphonylureas and biguanides. These differ not only in their chemical structure but also in their mechanism of action and safety over long term use.

Biguanides

Phenformin, butformin and metformin are some of them. Their primary mechanism of blood glucose lowering action is not on pancreas i.e. they don't increase insulin release or production. They act by increasing the utilization of glucose in tissues other than pancreas (extrapancreatic action) like liver and muscle, decreasing the formation of glucose (gluconeogenesis) in liver and reducing the absorption of glucose from intestines. The increased utilizaiton of glucose under the influence of biguanide drugs especially phenformin produces lactic acid causing lactic acidosis, a major and serious side effect. For this and other reasons, phenformin is discontinued but is used occasionally in some countries. In general, biguanides are not popular because of their side effects.

Sulphonylureas

The earlier drugs of this type are tolbutamide (Orinase), chlorpropamide, acetohexamide (dymelor) and tolazamide (tolinase). Their primary site of action is directly on the pancreas on the insulin producing beta cells in the islets of Langerhans. Their action is therefore by increasing the release of insulin which in turn reduces blood glucose. They are also known to act on tissues other than pancreas (extrapancreatic effect) like liver. While tolbutamide has shorter duration of action of 6-10 hours, tolazamide has a longer duration of action upto 20 hours. The above drugs are replaced in recent times by newer drugs of the series like glibenclamide and glipizide referred to as second generation sulphonylureas. Glibenclamide is 50-100 times more potent than tolbutamide. Its duration of action is even upto 24 hours. Because of this property, glibenclamide treatment more commonly results in hypoglycemia (fall in blood glucose level below normal). The potency and duration of action of glipizide is similar to glibenclamide. Side effects of second generation sulphonylureas are less and therefore are more widely used. However, myocarditis is reported in some obese diabetics treated by sulhonylureas. Other side effects seen are hyponatremia, transient leukopenia and thrombocytopenia.

Disadvantages of the Present Drugs

All these drugs are to be taken daily or sometimes more than once in a day. Their regular use for a number of years is associated with many acute and chronic side effects which are sometimes serious. Strict follow up of dietary, exercise and therapeutic schedule is necessary without which hypoglycemia or hyperglycemia, diabetic ketoacidosis may occur. Daily injection of insulin besides being painful may cause depression and other psychological problems to the patients. Because biguanides cause lactic acidosis, they are not in regular use in many countries. Chronic use of sulphonylureas may cause severe hypoglycemia, coma, allergic reactions, leukopenia, skin and gastrointestinal disturbances which predispose to coronary artery diseases. Chronic usage might also necessitate increase in dose.

Medicinal Plants: A Better Choice

There is considerable interest in recent years on natural drugs from medicinal plants, the nature's gift to mankind. Their superiority over the above mentioned chemical drugs is established by the well recognized and documented fact that most of the plant drugs are either free from or have only fewer side effects. Plant products for the treatment of diseases including diabetes mellitus have been in use from time immemorial in almost all the countries of the world with rich traditions of ancient medicine. The ancient system of Ayurvedic medicine of India is acclaimed to be one such great system of medicine. Many plants or Ayurvedic preparations are available which have been described by great ancient Indian Rishis like Charaka and Sushruta in their treatises. No attempt has been made to give here a list of all those medicinal plants which are useful in diabetes.

### SUMMARY OF THE INVENTION

In one aspect, the present invention relates to a method of preparing an anti-diabetic extract from banyan bark comprising: contacting crushed banyan bark with acetone to create a mixture having a solid phase and a liquid phase; separating said solid phase of said mixture from said liquid phase of said mixture; concentrating said liquid phase of said mixture; and purifying said concentrated liquid phase by chromatography and obtaining a light yellow product.

The invention also relates to a method of treating a mammal with a high blood glucose level comprising: identifying a mammal in need of such treatment; providing a banyan bark extract obtained by the above method; and administering said banyan bark extract to said mammal. Furthermore, the invention relates to a pharmaceutical composition comprising a banyan bark extract obtained by the above method, or a pharmaceutically acceptable salt thereof, and a pharmaceutically acceptable diluent, excipient, or carrier.

### DETAILED DESCRIPTION OF THE PREFERRED EMBODIMENTS

Banyan Bark: Its Known Uses and Disadvantages of its Earlier Preparations

Extracts of two species of trees belonging to the same genus Ficus namely Ficus bengalensis and Ficus glomerata possess antidiabetic activity. We chose the more common one Ficus bengalensis known as banyan tree.

F. bengalensis is a very large perenial tree growing to a height of about 30 meters, sending down many aerial roots from the branches. This extends the growth of the tree indefinitely. This tree belongs to the family Urticaceae and grows all over India.

Earlier Work on Hypoglycemic Activity of Banyan Tree bark.

Aqueous extract of the bark of F. bengalensis was found to reduce blood sugar in normal as well as alloxan diabetic rabbits. Adequate period of fasting seems to favour absorption and action of the extract better.

Three flavonoid compounds were isolated from ethanolic extract of the bark. Out of the three, a glycoside called bengalinoside produced maximum fall in blood sugar in normal and mildly diabetic rabbits. The compound was only half as active as tolbutamide. It did not have any effect on severely diabetic rabbits with fasting blood glucose (FBG) of 250 mg/dl or above (Augusti 1975). Leucocyanidin 3-0-β D-galactosyl cellobioside (dark green) and pelargonidin 3-0-α-rhamnoside (yellow red) isolated from the ethanol extract of the bark of F. bengalensis (Subramanian and Misra 1977) were found to have antidiabetic effect either by a single dose of 250 mg/kg or on long term treatment with 100 mg/Kg/day in alloxan induced diabetic (mild but not severe diabetes) rats (Cherian et. al. 1992). Leucocyanidin has insulin sparing action.

Hypoglycemic and insulin secreting effect of water extract of F. bengalensis was also reported (Achrekar et. al. 1991). There is no report of any pure compound isolated either from the water extract or ethanol extract of banyan bark active in severe diabetes.

Disadvantages of Earlier Banyan Tree Bark Compounds

The glycosides isolated from the ethanol extract of the banyan bark were active only in alloxan induced mild diabetes in rats and rabbits. The above compounds were active even in mild diabetes at a higher dose of 100 mg/Kg/day. The above compounds were not active in severe diabetes. Our preliminary studies revealed that the ethanol extract of the bark of banyan tree contains blood glucose increasing/hyperglycemic compounds which are undesirable for diabetic patients.

Our Product from Banyan Tree Bark and its Advantages

The methods of the present invention reveal that the ethanol extract of the bark of banyan tree contains one or more blood glucose increasing compounds, which are undesirable to a diabetic patient. We therefore devised a method to remove the undesirable blood glucose increasing compounds from the extract of the bark of this plant. Instead of ethanol extract, we used acetone extract which gave a compound with better activity. Our purified banyan bark compound ACS (acetone soluble) is active not only in mild but severe alloxan induced diabetes in rabbits. It is active at a much lower dose of even 15 mg/Kg in mild and 25 mg/Kg in severe diabetes. It has longer lasting effect and gives scope for intermittent therapy instead of daily therapy. At the end of therapy, no toxic affects were seen.

### EXAMPLES

### Example 1

### List of Raw Materials and Chemicals

Banyan tree (Ficus bengalensis) bark, silica gel G. Sephadex LH-20 (25-100 μ size), n-hexane, acetone, glucose, cholesterol, kits for estimation of glucose, cholesterol and triglycerides, methanol and ethyl acetate. Insulin assay kit if serum insulin level is estimated. Alloxan or streptozotocin for inducing diabetes.

Animals: Rabbits for alloxan diabetes. Rats for streptozotocin induced diabetes.

Utilities: Water, distilled (or deionized) water, electricity.

### Example 2

### List of Equipment

Extractor vessel; Glass or polypropylene columns for chromatography (2″×4 ft); Deionizer for deionized water; Glass flasks, beakers, water baths; Magnetic stirrers; Spectrophotometer (UV-Visible) recording; High performance liquid chromatography instrument with Zorbax ODS (C18) (Octadecyl silane) column or equivalent; Deep freeze (−40° C.); Refrigerator; Cold room.

### Example 2

### Preparation of the Antidiabetic Banyan Bark Compound ACS

Step 1: Preparation of the Bark for Extraction.

The bark of Ficus bengalensis was collected from trees situated in different parts of Delhi. Fresh bark was cleaned by peeling off the outer green layer and dried under sunlight. The bark was cut into pieces and ground in a hand grinder.

Step 2: Preparation of Acetone Extract

100 g of fresh banyan bark cleaned, dried, cut and powdered as above was extracted many times (till exhaustion) with several volumes of acetone. The acetone extract was evaporated to dryness in vacuum and washed with several volumes of hexane. Hexane washings (inactive) were discarded. 1 g. of the residue was purified at a time by chromatography on silicic acid column.

Step 3: Silicic Acid Chromatography of Acetone Extract

100 gm of silica gel (60-120 mesh) was washed successively with several volumes of distilled water and methanol, dried in the air and activated at 110° C. for 72 hours. The activated silica gel was filled into a column (50×2.5 cm) as a slurry in acetone. Packing of the column was then accomplished by passing acetone at a flow rate of 1 ml/minute for 2-3 hours and gently tapping the column during this period until the gel bed stops descending. Elution was done by gravity feeding and constant flow rate was maintained with the help of a Mariotte flask.

1 g. of the dried acetone extract was redissolved in 10 ml of acetone and applied on the silica gel bed by underlayering method (sample solution by virtue of its higher density settles at the bottom when introduced into the pure solvent). Elution was started at this stage at a flow rate of approximately 1 ml/minute. The column was connected to a Mariotte flask containing acetone. The sample solution was allowed to completely soak into the gel bed. A flow rate of 1 ml/min. was accurately maintained thereafter. Initial volume of 150 ml of the eluent was discarded. A second fraction of 300 ml of the eluent with yellow color was then collected. This fraction which was active (see below) was evaporated to dryness and was designated as “silica gel fraction”. The Mariotte flask was then removed and elution was done until the remaining acetone was eluted. The column was connected to a Mariotte flask containing methanol and eluted with methanol at a flow rate of 1 ml/min. 300 ml of the eluent was thus collected and evaporated to dryness. This methanol fraction was inactive and discarded.

The ‘silica gel fraction’ obtained above was suspended in water and tested for activity by glucose tolerance test (GTT). It showed good hypoglycemic effect. In a dose of 50 mg/kg body weight it prevented the rise in blood glucose level seen in the same rabbit before treatment. The improvement in glucose tolerance was remarkable. At the end of 2 hours, the blood glucose was only 157 mg % as compared with 275 mg % recorded before treatment. Thus there was 43% reduction by a smaller dose of 50 mg/kg.

Thin layer chromatography on silica gel G glass plates with ethylacetate: methanol water (100:16.5:13.5 V/V) as eluent, however, revealed that the fraction is still not homogeneous. This fraction separated into 3 bands. So it was further purified on lipophilic sephadex LH-20 as indicated in step 4 below.

Step 4: Purification of the Silica Gel Fraction using Sephadex-LH-20

20 grams of sephadex-LH-20 was swollen in 150 ml of methanol for 48 hours, heated to boiling, cooled and filled into a column (50×2.5 cm). Packing of the column was accomplished by passing methanol at a flow rate of 1 ml/min. for 24 hours. 150 mg of silica gel fraction was dissolved in 1 ml of methanol and applied on the gel bed by underlayering method and eluted with methanol. 5 ml samples were collected at a flow rate of 0.5 ml/minute. During elution three bands were clearly visible. A small yellow band came out first (Fraction 1). After elution of this minor yellow fraction (not active) the major yellow band (Fraction 11) was collected as a single fraction. Usually it was eluted in about 80 fractions (5 ml each). This showed hypoglycemic activity. The total organic content of each sample was estimated by dichromate oxidation method. Elution was continued with methanol at a flow rate of 1 ml/min. for 24 hours till the pink inactive compound (third band) was eluted (Fraction-III). For regeneration of sephadex, the sephadex was then eluted with dilute ammonia (6 ml of 25% ammonia in 100 ml followed by 50% methanol and then methanol. The procedure is shown in Flow Chart I.

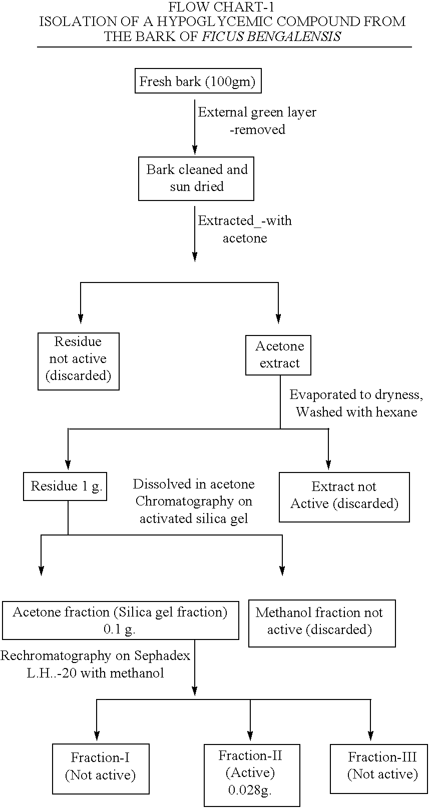

As stated above pooled fractions of peak-II only showed hypoglycemic activity. In the glucose tolerance test, at the end of 2 ½ hours, the blood glucose level after oral glucose load was only 130 mg % when compared with 238 mg % in the untreated animals. The reduction was 45%. It is to be emphasized that in this test each rabbit served as its own control because GTT was done in the same diabetic rabbit before and after treatment. The dose of the sephadex-LH 20 purified fraction used in these experiments was only 15 mg/kg body wt. The active sephadex purified fraction is referred to as sephadex fraction hereafter.

Purity of the Product

Since we isolated another water soluble hypoglycemic compound from banyan bark the acetone soluble compound purified by the above method is called banyan bark compound ACS.

The purity of this factor was tested by thin layer chromatographic technique on glass plates 25×25 cm coated with silica gel 0.5 mm thick using the solvent system ethylacetate: methanol: water 100:16.5:13.5 v/v. It showed only one peak. However when separated by high performance liquid chromatographic technique in Waters HPLC system using C-18 reverse phase column it showed a major peak (FIG. 1) and a very minor peak. Attempts to remove the minor peak were not successful. Due to non availability of preparative HPLC apparatus it could not be purified further. Thus the hypoglycemic compound purified is nearly homogenous (about 95% pure) except for a very minor impurity which can also be removed by HPLC technique. The degree of purification achieved is shown in table 2. Although there appears to be nearly 6 fold purification it has to be pointed out that it is active at a dose of only 15 mg/kg body weight in subdiabetic rabbits which is much less than the dose required by the standard drug tolbulamide.

TABLE 2
Hypoglycemic activity at different stages of purification. Specific
activity expressed as percent fall in blood glucose levels per mg of
the purified fraction 2.5 hours after glucose load.

| | | | Percent fall i.e. | |
|---|---|---|---|---|
| | | | improvement in | |
| Fraction tested | Dose employed | | glucose tolerance | Specific activity |
| Acetone extract | 130 | mg/kg | 47.5 | 0.52 |
| Silica gel fraction | 50 | mg/kg | 43 | 0.86 |
| Sephadex fraction | 15 | mg/kg | 45 | 3.0 |
| Alcohol extract | 1 | g/kg | 44 | 0.044 |

For comparison the specific activity of the alcoholic extract was given at the end.

### Example 3

### Properties of the Purified Banyan Bark compound ACS

The following are some of the physical properties of the compound: The compound is light yellow in color. It is soluble in acetone and methanol and insoluble in hexane, benzene and water. It has absorption at 205.8 and 258-264 nm wave length in the UV region. The compound produced bluish black color on the addition of ferric chloride solution which is one of the properties of phenolic compounds. On the addition of alkali (methanolic KOH) its color changes to red.

Stability

The compound is reasonably stable. When stored at −4° C., its activity was stable up to at least one month.

### Example 4

### Product Specifications

Chemical and Physical

The compound is a light yellow substance soluble in acetone, methanol and insoluble in water, hexane, benzene. It shows absorption maxima at 205.8 and 258-264 nm. 268 and 340 nm. Addition of methanolic potassium hydroxide to water suspension containing the compound changes the color to red. Addition of ferric chloride solution to water suspension containing the compound produces bluish black color. Thin layer chromatography on silica gel G plates with ethylacetate:methanol:water (100:16.5:13.5 V/V) as mobile phase shows a single band.

Animals:

The drug is tested in two groups of five male rabbits each weighing about 1 to 1.5 kg. One group was given water suspension of purified banyan bark compound while the other received same volume of water.

### Example 5

### Induction of Diabetes

Rabbits were fasted overnight. Alloxan solution was prepared freshly in 0.7% sterile NaCl, the pH adjusted to 4.5 with solid citric acid (in order to compensate the osmolarity of alloxan) and kept in ice bath. The solution should be used within half an hour of its preparation. Alloxan solution equivalent to 80 mg/kg body weight (b.w) of each rabbit was administered intravenously through the marginal ear vein of rabbits. Food was provided four hours after alloxan injection. Urine sample was tested for glucose by Qualitative Benedicts regent. To 5 ml of this solution 8 drops of urine were added and boiled for 2 minutes. Green or yellow color or orange to reddish precipitate is an indication of diabetes in the animals. Blood glucose was also determined (see below). Increased blood glucose in urine is an indication of diabetes in the animals.

### Example 6

### Blood Glucose Determination

Fasting blood glucose was estimated once a weak for one month. Rabbits with consistently abnormal fasting plasma glucose values of 150-200 mg/dl were used. Plasma glucose was estimated with enzymatic kit (glucose oxidase/peroxidase) of M/S Ranbaxy Diagnostics, New Delhi. Working solution (prepared as described in the kit) was mixed with 10 ml of either water (for blank), standard glucose (provided in the kit) or plasma (for test) and incubated at 37° C. for 15 minutes. Intensity of red colour was measured by its absorbance at 505 nm. Plasma glucose of the test solution was calculated from the optical density values of standard glucose (minus blanks) and test sample (minus blank) using the formula given in the instructions of the manufacturer.

Glucose oxidase kit from any good manufacturer can be used by following the instructions given by them.

### Example 7

### Glucose Tolerance Test

2 ml blood was collected in clean tubes containing suitable anticoagulant from overnight fasted diabetic rabbits. Water suspension of purified banyan bark compound ACS (15 MG/Kg b.w for subdiabetic rabbits or 25 mg/kg b.w. for mildly diabetic rabbits) was administered orally by Ryle's tube. After 90 minutes, 2 ml blood was again drawn. The plasma glucose value of this sample of blood gives an idea of the effect of banyan compound 90 minutes after its administration on the fasting blood glucose. This blood sample also serves as the zero hour sample in the glucose tolerance test. Then the rabbits were given glucose (2 g/kg.b.w) in 15 ml water orally. Blood samples were collected after one and 2.5 hours. Plasma was separated by centrifugation at 1000 rpm at room temperature for 10 minutes. Hemolysed samples were rejected. The supernatant plasma was stored in the refrigerator until use. Plasma glucose was estimated as early as possible after the collection of the 2.5 hour blood sample.

### Example 8

### Activity of the Test Sample

Purified banyan bark compound ACS brings about fall in plasma glucose in the zero hour (fasting), 1 hr and 2.5 hr. samples when compared with those in the untreated group (which received water only in place of Banyan bark ACS) of rabbits. The fall is more marked at 2.5 hr plasma glucose values. The average fall in 2 hr plasma glucose value produced by a single dose of 15 mg/kg b.w. in subdiabetic rabbits was about 35-40%; in mildly diabetic rabbits a single dose of 25 mg/kg b.w. produced 25-30% fall when compared with the values at 2.5 hrs of the untreated rabbit(s). Any batch of preparation which does not show this fall should be rejected.

Alternately diabetes can be induced in rats by streptozotocin by standard methods for testing the potency of each batch. But we preferred and used only alloxan diabetic rabbits.

### Example 9

### Antidiabetic Activity of Purified Banyan Bark Compound; ACS

Induction of Diabetes in Experimental Animals

Before any drug can be used in humans, it is obligatory to carry out extensive studies and demonstrate in animals that it is effective in diabetes and safe. In the assessment of efficacy of drugs in animals, there are many hurdles such as differences from humans in disease pattern, variation in absorption, metabolism and excretion and adverse reactions (side effects). No animal model of diabetes corresponds exactly to diabetes in humans. Nonetheless, experimental studies in animals have contributed a lot for the development of new antidiabetic drugs. Of the various animals, diabetes induced in rabbits is closer to human diabetes. But rats and mice (especially genetically diabetic mice) are also preferred by some.

Diabetes can be induced in animals by various methods. Destruction of beta cells in islets of Langerhans in the pancreas of animals is the most commonly used one. The chemicals which are widely used are alloxan in rabbits and streptozotocin in rats or mice. Streptozotocin induced diabetes is preferred now a days because it produces in rats diabetes resembling NIDDM of humans. Further, alloxan causes renal damage. However, many drugs which are now in use were developed after testing them in alloxan induced diabetes. The renal damage by alloxan is reversible over a period of time. Alloxan induced diabetes is still in use. We preferred alloxan induced diabetes in rabbits.

Alloxan Induced Diabetes in Rabbits

We produced in rabbits three types of diabetic state which are some what similar to sub-diabetic, mild and severe diabetic states in humans. Normal rabbits weighing about 1-1.5 kg were acclimatized to laboratory conditions and rabbit pellet diet of Hindustan Levers, Bombay. Alloxan solution was freshly prepared in 0.7% saline and pH adjusted to 4.5 with solid citric acid, kept in ice and used within half an hour. Intravenous injection of alloxan 80 mg/kg was administered through marginal ear vein. If any of the rabbits showed signs of hypoglycemia (in 6-8 hours) due to sudden release of insulin, 25% glucose solution was given orally by gastric intubation. Food was given 4 hours after alloxan injection. Blood glucose was tested at intervals of 5 days. Urine was also tested for the presence of glucose. Only rabbits with stabilized diabetic state (see below) were used for the experiments. Usually after 30 days of alloxan injection, the animals reached a stabilized state of diabetes. Then they were arbitrarily divided into three groups: (1) Subdiabetic, (2) Mildly diabetic and (3) Severely diabetic rabbits.

Subdiabetic Rabbits

In some of the rabbits, the fasting blood or plasma glucose levels which initially increased after alloxan injection, gradually came down to normal or slightly elevated values (upto 120 mg/dl). But these rabbits showed abnormal glucose tolerance with plasma glucose values which took much longer time than the usual 2 hours to return to normal after oral glucose load. This condition in rabbits is similar to the subdiabetic state in humans.

Mildly Diabetic Rabbits

Rabbits with elevated fasting plasma glucose (FPG) or fasting blood glucose (FBG) levels in the range (arbitrarily chosen by us) of 120-250 mg/dl were considered to be mildly diabetic. Their glucose tolerance was abnormal with plasma glucose values which remained much higher than normal even 2.5 hours after oral glucose load. This is similar to NIDDM in humans.

Severely Diabetic Rabbits

Rabbits with FPG or FBG values above 250 mg/dl were considered to be severely diabetic. When Glucose Tolerance Test (GTT) was performed, most of the animals died. So oral GTT was avoided in this group of rabbits. Severely diabetic rabbits with FPG values closer to 400 mg/dl are similar to IDDM patients.

### Example 10

### Assessment of Hypoglycemic (Antidiabetic) Activity: Fasting Blood (plasma) Glucose and Glucose Tolerance Test

In subdiabetic and mildly diabetic rabbits, after overnight fasting, blood was drawn in the morning from marginal ear vein. The Banyan bark compound ACS was given orally by gastric intubation at the dose indicated in each experiment. After 90 min. blood was again drawn (this sample gives an idea of the effect of the drug on fasting blood glucose in 90 min) and glucose tolerance test was performed by giving glucose (2 g/kg) solution orally. Blood samples were again drawn 1 and 2.5 hours after glucose. Plasma was separated and glucose determined. In untreated subdiabetic and mildly diabetic rabbits the plasma glucose values remain at higher than normal levels for a much longer time. However, treatment with an effective antidiabetic drug will bring down or normalize the plasma glucose levels depending on its efficacy, dose, duration of treatment and severity of diabetes.

Since the plasma glucose levels are nearly normal or only slightly elevated in sub-diabetic rabbits, after treatment with an antidiabetic drug there may or may not be any change in FPG. In fact fall in FPG is not desirable in such rabbits. But there would be improvement in glucose tolerance. So improvement in glucose tolerance is used as a parameter for antidiabetic effect of the drug under test in subdiabetic rabbits. In mildly diabetic rabbits which have elevated blood glucose levels, assessment of the antidiabetic activity is by fall in FPG as well as improvement in glucose tolerance.

In severely diabetic rabbits, the assessment was by fall in FPG value after treatment. Glucose tolerance test can't be used as a parameter in severely diabetic rabbits because the animals might die after oral glucose load.

### Example 11

### Estimation of Blood (Plasma) Glucose

Plasma glucose was determined as a measure of blood glucose. Plasma glucose was estimated by using glucose oxidase (since it is specific for glucose) coupled with peroxidase as described above using kits.

Studies with the Sephadex Fraction

a) Glucose Tolerance Immediately after the Administration of the Drug.

A group of 5 subdiabetic rabbits was subjected to glucose tolerance test. Then a single dose of 20 mg/kg of the sephadex fraction was orally administered to these rabbits after overnight fasting. Within 3 minutes glucose solution was given orally and GTT was performed to get an idea of the immediate effect of the drug on glucose tolerance.

TABLE 3
Immediate effect of banyan bark compound ACS
on glucosetolerance in subdiabetic rabbits.

Blood Glucose mg/dl

| | 0 Hr | 1.5 Hrs | 2.5 Hrs | % fall |
|---|---|---|---|---|
| Untreated | 105 | 305 | 285 | 7 |
| Treated | 95 | 267 | 191 | 32 |

Values represent mean of 5 rabbits.

The results in table 3 show that the banyan bark compound ACS has favorable action. The effect is however less pronounced as peak values of 267 mg % of blood glucose was seen also in treated animals but there was improvement as a whole because the FBG value at 2.5 hrs was 32% less than that in the untreated controls.

Longer Lasting Effect of Single Dose on GTT

It was also intended to see how long the effect of a single dose of the purified drug lasts. A single dose of 20 mg/kg body weight of the sephadex purified fraction of acetone extract of banyan bark was given and GTT performed 90 minutes later. The GTT was repeated at intervals of 15 days for 1 month. The results shown in table 4 indicate that the improvement of glucose tolerance (suppression of the GTT pattern by 34%) was seen upto 15 days. Even upto one month there was 10% suppression of GTT.

TABLE 4
Longer lasting effect of sephadex fraction of acetone extract
of banyan bark on glucose tolerance in subdiabetic rabbits.

| | Total blood glucose | |
|---|---|---|
| Days | (mg %) during GTT* | % fall |
| Initial | 1492 | |
| 90 minutes after drug | 1125 | 18 |
| 15 days after drug | 976 | 34 |
| 30 days after drug | 1252 | 10 |

Total blood glucose values in the table represent the sum of all the values of blood glucose at 30, 60, 90 120 and 150 minutes.

Studies on the Longer Lasting Hypoglycemic Effect with Multiple Doses of the Drug

In the studies mentioned above single dose of sephadex fraction showed improvement in glucose tolerance for a longer time. It was intended to try the effect of multiple doses of the drug on the improvement of glucose tolerance. However due to non-availability of the sephadex fraction in large amounts, acetone extract was only used in these experiments.

Subdiabetic rabbits were subjected to GTT 30 days after the alloxan treatment. One week after the last GTT, acetone extract (130 mg/kg) was orally administered to a group of 6 overnight fasted AR rabbits. GTT was performed 90 minutes later. A second dose of 130 mg/kg was administered orally 5 days after the first dose again after overnight fasting. This was followed 90 minutes later by a glucose tolerance test. GTT of these rabbits was tested again on the 5^{th} and the 13^{th} day after the second dose of the acetone extract. A control group of 5 rabbits were also subjected to glucose tolerance tests along with the treated group during the course of these studies. The results indicated that there was 33% improvement in the glucose tolerance 90 minutes after the second dose and the effect persisted even on the 13^{th} day.

Studies on the Acute Effects of the Silica Gel Fraction on the FBS Levels of Severely Diabetic Rabbits

Rabbits with FBS levels of 400 mg % and above were employed in the present studies. In these studies due to non-availability of sufficient amount of the sephadex purified material, studies were carried out with silica gel column purified material. It was orally administered to a group of 5 severely diabetic rabbits after overnight fasting. Fasting blood and blood samples at 1 hour intervals upto 4 hours following the administration of the drug were collected and analysed for glucose levels. A control group of 5 severely diabetic rabbits were subjected to similar treatment after administering 20 ml of distilled water instead of the drug. Single dose did not produce any fall in FBG upto 4 hours.

Delayed Hypoglycemic Effect of the Silica Gel Fraction

Since single dose did not have any immediate effect on FBG, it was felt that perhaps still longer time might be necessary in the case of severely diabetic rabbits. Therefore, another experiment was carried out in which the sephadex purified fraction was given as a single dose (50 mg/kg) to severely diabetic rabbits. But the fasting blood sugar values were determined at intervals of 2 days up to 8 days. As is evident from table 5 the drug has lowered the FBS of severely diabetic rabbits by 33.7% at the end of 2 days. Further some effect was still evident even after 4 days. The FBS however reached to prior treatment levels by the 8^{th} day.

TABLE 5
Delayed effect of a single dose of silica gel fraction of
acetone extract of banyan bark on FBS in severely diabetic rabbits

Fasting blood glucose mg %

| Days | Untreated | Treated |
|---|---|---|
| 0 | 405 | 426 |
| 2 | 418 | 281 |
| 4 | 492 | 398 |
| 8 | 486 | 502 |

Values represent mean. of 5 severely diabetic rabbits.

The interesting observation in the experiment is the delayed effect of the drug. The drug does not act as such. Probably it gets metabolized and one of the metabolities brings about the fall in fasting blood sugar level by acting directly on glucose metabolism in liver, muscle etc. The metabolism of the drug in severely diabetic rabbits perhaps takes longer time than in sub diabetic rabbits. The second possibility is that the drug stimulates a very few surviving beta cells of the pancreas in the severely diabetic rabbits which is a time taking process.

Whatever may be the explanation the fact that it has hypoglycemic even in severely diabetic rabbits confers a distinct advantage to this drug. Example 12

### Mechanism of Action of Banyan Bark Compound ACS

Mechanism of Action:

Antidiabetic or hypoglycemic drugs act by more than one mechanism. One is pancreatic (beta cryotropic) action which is by stimulating the release and or synthesis of insulin. Insulin stimulates metabolic pathways of glucose utilization and thereby reduces blood glucose level. The stimulation of pancreas to release insulin is possible only if there is functional pancreas but not otherwise. The second method is by the direct action of the drug on the metabolic pathways of carbohydrate, lipid and protein metabolism in tissues other than pancreas like liver, muscle and adipose. This is extra pancreatic effect.

Effect of Banyan Bark Compound ACS on the Serum Insulin Levels During GTT of the Subdiabetic Rabbits

In subdiabetic rabbits, GTT was performed. Samples were drawn at 30 minutes intervals up to 150 minutes after the glucose load, for the analysis of glucose and levels of insulin in the serum. One week later 60 mg/kg of the silica gel purified fraction was orally administered as a water suspension to rabbits after overnight fasting. Glucose tolerance test was performed 90 minutes later by the method described earlier. Fasting blood samples and blood samples 90 minutes after the drug and 30, 60 and 150 minutes after the glucose load were collected for the analysis of blood glucose and serum insulin levels. Serum insulin was measured by radioimmunoassay using the kit purchased from Bhabha Atomic Research Centre, Bombay.

From Table 6 it is evident that the drug has enhanced the serum insulin levels in 90 minutes after its administration. Peak levels, however were reached 30 minutes after the administration of glucose. This clearly shows that the compound brings about its hypoglycemic effect by stimulating pancreatic beta cells for the release of insulin. The serum insulin levels corresponded well to the blood glucose levels in the glucose tolerance pattern. This shows that improvement in glucose tolerance in subdiabetic rabbits is due to the release of insulin into the blood.

TABLE 6
Effect of banyan bark compound ACS on the serum
insulin levels during GTT in subdiabetic rabbits.

| | | Seurm insulin level (log cpm)* | | |
|---|---|---|---|---|
| Time | | Untreated | Treated | Normal |
| Initial | | 3.08 | 3.08 | 2.093 |
| 90 | minutes after drug | 3.07 | 3.0 | 2.1 |
| | (0 time for GTT) | | | |
| 30 | minutes after glucose | 3.08 | 2.98 | 2.86 |
| 60 | minutes after glucose | 3.06 | 3.05 | 2.81 |
| 150 | minutes after glucose | 3.16 | 3.10 | 3.02 |

The values represent log counts per minute in the radioimmunoassay.

Possible major site of action of the active compound appears to be the pancreas which is stimulated to release more insulin following oral glucose load. This is a property shown by many drugs in use today or reported by others. Since the banyan compound was active in severe diabetes also, it seems to have extra pancreatic effect. The effect of the drug on tissue glycogen and protein content was also assessed.

Studies on the Effect of the Drug on Tissue Glycogen and Protein Levels of Subdiabetic Rabbits

Subdiabetic rabbits produced by the method described earlier were divided into two groups of 5 animals each. Rabbits of group-I were treated with the sephadex fraction by the following regimen.

A total number of three doses were administered. First two doses of 50 mg/kg body weight each were administered on two successive days to rabbits (GTT was not done) of this group. 5 days later a third dose of 25 mg/kg body wt. was orally administered after overnight fasting. Six hours after the last dose of the drug blood samples were drawn for the analysis of serum urea. The animals were then sacrificed and liver, heart and muscle were collected for the analysis of glycogen and total protein contents.

Estimation of Tissue Protein

The tissue (liver, heart and muscle) (as 1% homogenate) protein content was assayed by the method of Lowry et. al (1951).

Estimation of Tissue Glycogen

Glycogen was isolated from the tissues and estimated by the method of Good et. al (1933).

The glycogen content of the muscle and liver slightly increased and the protein content of the liver, heart and muscle (table 7) remained unaffected by the treatment.

TABLE 7
Effect of the sephadex fraction on the tissue
glycogen and protein content of the rabbits. Course
of the treatment is described under methods.

| | | Glycogen gm/ | |
|---|---|---|---|
| | | 100 gm. tissue) | Protein mg/gm |
| Liver | Control | 0.68 | 162 |
| | Treated | 0.71 | 159 |
| Muscle | Control | 0.23 | 163 |
| | Treated | 0.25 | 168 |
| Heart | Control | — | 147 |
| | Treated | — | 143 |

### Example 13

### Toxicity Studies

In order to find out the effect of the drug on liver function, serum urea was estimated by using kits.

In order to find whether higher doses of the drug produce any adverse effect on liver, one group of 5 subdiabetic rabbits was given 50 mg/Kg b.w. (slightly more than 3 times the effective dose) daily once in the morning for 3 days. Three doses of the drug were given. Control group of animals received water in place of banyan compound. Blood urea levels were estimated. Blood urea levels were estimated in treated and untreated animals. The blood urea levels were 32 mg % and 40 mg % in control and treated animals respectively. Thus treatment of subdiabetic rabbits with 3 higher doses of the drug over a period of one week did not affect blood urea levels which reflect the liver function. These results give only a preliminary idea that this drug is not likely to be toxic to liver. Certainly more detailed acute and chronic toxicity studies, pharmacological and teratogenic studies are necessary before reaching the final conclusion that the drug is not toxic. The conclusions that however could be reached are that the drug improved glucose utilization by increasing serum insulin levels without changes in blood urea. The mechanism of action is similar to that of tolbutamide.

### Example 14

Hypolipidemic Effect of Banyan Bark Compound ACS

Two groups (5 each) of subdiabetic rabbits were used. One group of rabbits was treated on two successive days with 50 mg/kg b.w. of silica gel fraction and on the third day to overnight fasted rabbits 25 mg/kg b.w. of the drug was given. Six hours later blood was drawn for determination of serum lipids. The results in table 8 show that there was fall in the blood levels of cholesterol and triglycerides and increase in phospholipid following the treatment with 3 doses of the drug over a period of one week. This means that even with a short duration of treatment there was indication of hypolipidemic effect of the compound. Further detailed studies are necessary.

TABLE 8
Effect of the sephadex fraction of banyan bark on the serum
Lipid levels (triglyceride, phospholipids and cholesterol)
of the subdiabetic rabbits

| | Control | Treated animals | % fall |
|---|---|---|---|
| Triglycerides (mg %) | 149 | 99 | 66 |
| Phospholipids (mg %) | 125 | 185 | — |
| Cholesterol (mg %) | 108 | 78 | 28 |

### REFERENCES

The following references were referred to herein:

1. Achrekar, S; Kaklij, G. S; Pote, M. S. and Kelkar, S. M (1991). Hypoglycemic activity of Eugenia jambolana and Ficus bengalensis: Mechanism of action. In vivo 5, 143-147.

2. Augusti, K. T. (1975) Hypoglycemic action of bengalinoside, a glucoside isolated from Ficus bengalensis on normal and alloxan diabetic rabbits. Ind. J. Physiol, Pharmacol, 19. 218-220.

3. Cherian, S; Kumar, R. and Augusti, K. T. (1992). Antidiabetic effect of a glycoside of pelargonidin isolated from the bark of Ficus bengalensis. Ind. J. Biochem. Biophys. 29, 380-382.

4. Good, C. A; Kramer, H. and Somogyi, M (1933). The determination of glycogen. J. Biol. Chem. 100, 485.

5. Lowry, O. H; Rosebrough, N. J; Farr, A. O. and Randall, R. J.(1951), Protein measurement with folin phenol reagent. J. Biol. Chem. 193, 265.

6. Subramanian, M. P. and Misra, G. S. (1977) Chemical constituents of Ficus bengalensis. Ind. J. Chem 15B, 762-763.

What is claimed is:

1. A method of preparing an anti-diabetic extract from banyan bark comprising:
a) contacting crushed banyan bark with acetone to create a mixture having a solid phase and a liquid phase;
b) separating said solid phase of said mixture from said liquid phase of said mixture;
c) concentrating said liquid phase of said mixture; and
d) purifying said concentrated liquid phase by chromatography and obtaining a light yellow product.

2. The method of claim 1, wherein said purification step uses an eluent comprising acetone.

3. The method of claim 1, wherein said separating in step b) is filtering.

4. The method of claim 1, wherein said purifying is by using silica gel.

5. The method of claim 4, wherein said purifying is by using a silica gel plate.

6. The method of claim 4, wherein said purifying is by using a silica gel column.

7. A method of treating a mammal with a high blood glucose level comprising:
a) identifying a mammal in need of such treatment;
b) providing a banyan bark extract obtained according to claim 1; and
c) administering said banyan bark extract to said mammal.

8. The method of claim 7, wherein said mammal is a human.

9. A pharmaceutical composition comprising a banyan bark extract obtained according to claim 1, or a pharmaceutically acceptable salt thereof, and a pharmaceutically acceptable diluent, excipient, stabilizer or carrier.
